(19) United States
(12) Patent Application Publication
(10) Pub. No.: US 2015/0022372 A1
(43) Pub. Date: Jan. 22, 2015

(54) MEDICAL DATA ACQUISITION SYSTEMS AND METHODS FOR MONITORING AND DIAGNOSIS

(71) Applicant: Tesseract Sensors, LLC, Sanford, FL (US)

(72) Inventor: Michael J. Vosch, Titusville, FL (US)

(21) Appl. No.: 14/334,656

(22) Filed: Jul. 17, 2014

Related U.S. Application Data
(60) Provisional application No. 61/847,873, filed on Jul. 18, 2013.

(51) Int. Cl. A61B 5/00; A61B 5/0402; G08C 17/02

(52) U.S. Cl. CPC A61B 5/0002; G08C 17/02; A61B 5/0402
USPC 340/870.07

(57) ABSTRACT

Medical data acquisition systems and methods for monitoring and diagnosis are disclosed. According to an aspect, a system may include one or more electrodes configured to detect biological data and to convert the detected biological data into a signal. The system may also include a monitor recorder configured to receive the signal and store the detected biological data. Further, the system may include a transceiver configured to wirelessly communicate the biological data.

### CROSS REFERENCE TO RELATED APPLICATION

This application claim the benefit of and priority to U.S. Provisional Patent Application No. 61/847,873, filed Jul. 18, 2013 and titled MEDICAL DATA ACQUISITION SYSTEMS AND METHODS FOR MONITORING AND DIAGNOSIS; the content of which is incorporated herein by reference in its entirety.

### TECHNICAL FIELD

The present subject matter relates to medical monitoring and diagnosis. Particularly, the present subject matter relates to medical data acquisition systems and methods for monitoring and diagnosis.

### BACKGROUND

Medical data acquisition equipment has been used in many settings including hospitals. In other applications, such equipment can be used for remote monitoring of individuals. Example data that can be collected and remotely communicated for analysis includes electrocardiography data. It is desired to provide improved systems and techniques for medical monitoring and diagnosis. Particularly, it is desired to provide improvements for rapidly collecting large amounts of medical data, wirelessly communicating the data, and remotely analyzing the data.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Disclosed herein are medical data acquisition systems and methods for monitoring and diagnosis. According to an aspect, a system may include one or more electrodes configured to detect biological data and to convert the detected biological data into a signal. The system may also include a monitor recorder configured to receive the signal and store the detected biological data. Further, the system may include a transceiver configured to wirelessly communicate the biological data.

### BRIEF DESCRIPTION OF THE DRAWINGS

The foregoing summary, as well as the following detailed description of various embodiments, is better understood when read in conjunction with the appended drawings. For the purposes of illustration, there is shown in the drawings exemplary embodiments; however, the presently disclosed subject matter is not limited to the specific methods and instrumentalities disclosed. In the drawings:

FIG. 1 is a block diagram of an example medical data acquisition system for monitoring and diagnosis in accordance with embodiments of the present subject matter;

FIG. 2 is an exploded view of a medical data diagnostic system;

FIGS. 3 and 4 are bottom and top perspective views, respectively, of the system shown in FIG. 2;

FIG. 5 is another top perspective view of the system shown in FIG. 2;

FIG. 6 is a top view of the electronic circuitry of the system shown in FIG. 2;

FIG. 7 is a block diagram of another example medical data acquisition system for monitoring and diagnosis in accordance with embodiments of the present subject matter; and

FIGS. 8-11 show different examples of patch electrodes in accordance with embodiments of the present subject matter.

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies. Moreover, although the term “step” may be used herein to connote different aspects of methods employed, the term should not be interpreted as implying any particular order among or between various steps herein disclosed unless and except when the order of individual steps is explicitly described.

As an example, a system in accordance with the present disclosure may include a monitoring system card configured to store and transmit data received from multiple manners such as, but not limited to, an EKG strip or full 12 lead EKG. Further, a system may include a 2-8 channel circuit card capable of measuring biometrics and transmitting biometric data. The monitoring system card can perform numerous physiological measurements at a very high sampling rate. For example, the sampling rate may be up to 32,000 samples per second per channel. The card may be capable of reading, processing, and transmitting the following information in addition to EKG: microphone/voice recording, accelerometer, respirations, oxygen (O₂) saturation, and/or the like. In an example, the system may include a battery having a 1 week life or longer. Further, the card may include programmable intervals of data recording and/or event or patient activation.

A monitoring system card in accordance with the present subject matter may obtain patient data for software analysis as described in examples herein and in any suitable technique. Example techniques for obtaining and communicating patient data include, but are not limited to: via a BLUETOOTH® transmitter to an analysis station; data transmission via a BLUETOOTH® transmitter to a cloud network or to a cell phone (which in turn can transmit data over a data network) to an analysis station; and writing data to a micro SD card or data transmission via micro USB. Post processing can be defined by an end user.

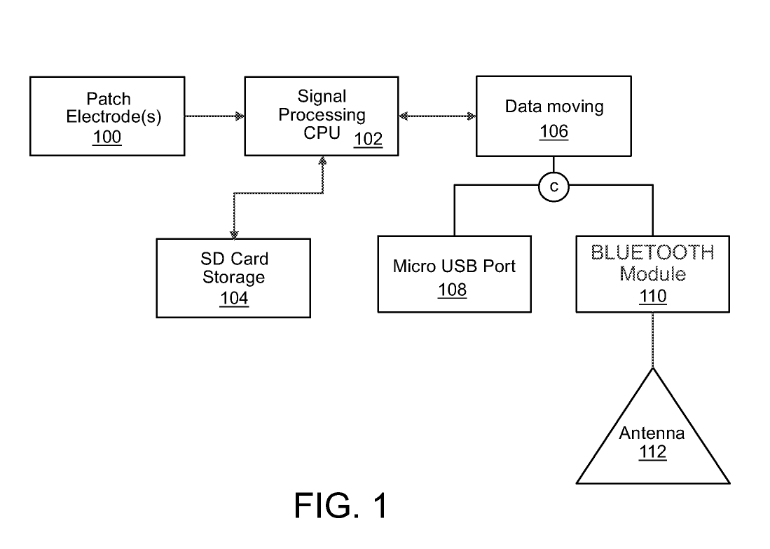

FIG. 1 illustrates a block diagram of an example medical data acquisition system for monitoring and diagnosis in accordance with embodiments of the present subject matter. Referring to FIG. 1, the system includes one or more patch electrodes 100 configured to detect physiological data and to convert the detected physiological data into a signal. For example, the patch electrodes 100 may be attached to a person for collecting electrocardiography (ECG) data. A patch electrode may be any suitable type of electrode or sensor configured to detect current or voltage. The patch electrode may include an adhesive for attachment to a patient. The detected current or voltage may indicate physiological information about the patient as will be understood to those of skill in the art.

The patch electrodes 100 may be communicatively connected to a signal processing central processing unit (CPU) 102 such that the ECG data and/or other physiological data can be communicated to the CPU 102. For example, the patch electrodes 100 may be suitably connected to the CPU 102 by one or more lead wires and conditioning circuitry. The data may be converted to a signal for communication to the CPU 102. The CPU 102 may process, organize, and store the ECG data in secure digital (SD) card storage 104.

A data moving module 106 may receive ECG data from the CPU 102 and pass the data to either a micro USB port 108 or a BLUETOOTH® module 110. The data communicated to the USB port 108 may be suitably downloaded by a computing device, such as a laptop computer. The module 110 may wirelessly communicate the data by use of an antenna 112 or transceiver.

Data downloaded via the USB port 108 or received from the antenna 112 via wireless communication may be evaluated by a software analysis system. The software analysis system may import the data in one of various file formats including, but not limited to: SIFOR file format (SDF), simple control protocol (SCP), medical diagnostic workstation (MDW) (for use with Cardio Perfect ECG Diagnostic System), MIT format, and 2-10 cubed (Phillips format).

In accordance with embodiments of the present disclosure, the system may utilize software for receiving and processing data collected from an individual as described herein. For example, the software residing on a system shown in FIG. 1 may be implemented by the CPU 102. The software that receives the obtained data can be any suitable ECG diagnostic system software. The data produced by the card can be formatted with the CPU 102 so that the receiving software can read the data in, in accordance with that software's particular requirements. This allows the card to be developed to meet a myriad of systems while maintaining the same configuration, thereby reducing overall cost to manufacture different models for different external software systems.

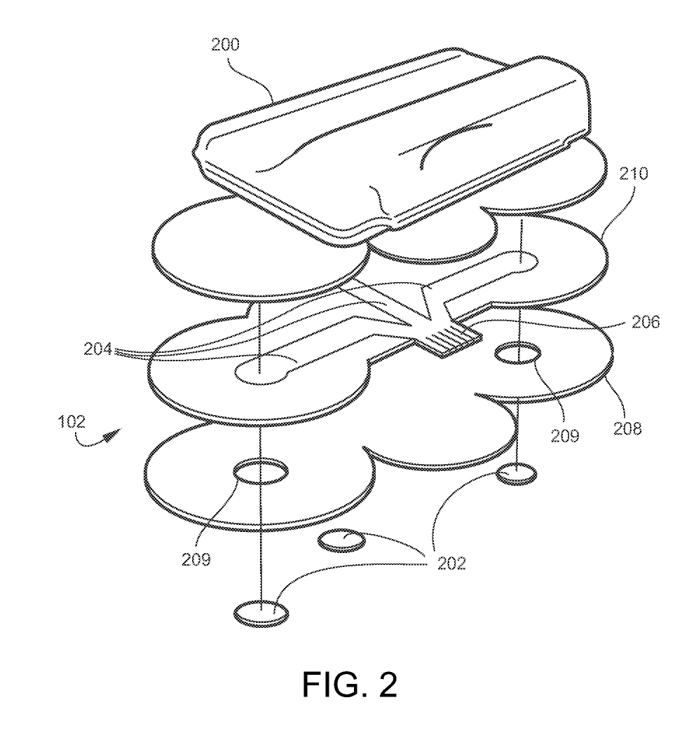

FIG. 2 illustrates an exploded, perspective view of a medical data diagnostic system in accordance with embodiments of the present disclosure. Referring to FIG. 2, the system may include a plastic protective shell or casing 200 for containing electronics such as the components shown in FIG. 1. For example, the casing 200 may contain the CPU 102, the SD card storage 104, the data moving module 106, the USB port 108, the module 110, and the antenna 112 shown in FIG. 1. The casing 200 may be made of any suitably rigid material such as plastic or metal. This material can be various forms of ABS, carbon fiber, or metal composites that can allow for the ease of manufacture at a low cost with a high reliability for the end-user.

The system may include patch electrodes, generally designated 102. The patch electrodes 102 may include multiple electrodes 202 that are connected to conductive lines or leads 204 for electrical communication with an interface 206. The interface 206 may connect, for example, the patch electrodes 102 with the CPU 102 shown in FIG. 1 and operate in accordance with examples disclosed herein.

Further, the system include an adhesive component 208 for attachment to a patient. The adhesive component 208 may define holes 209 for containing or holding the electrodes 202. The adhesive component 208 may attach on a top side to a layer 210 that holds the leads 204. The system may also include another layer 212 for interfacing the casing 200 and the layer 210.

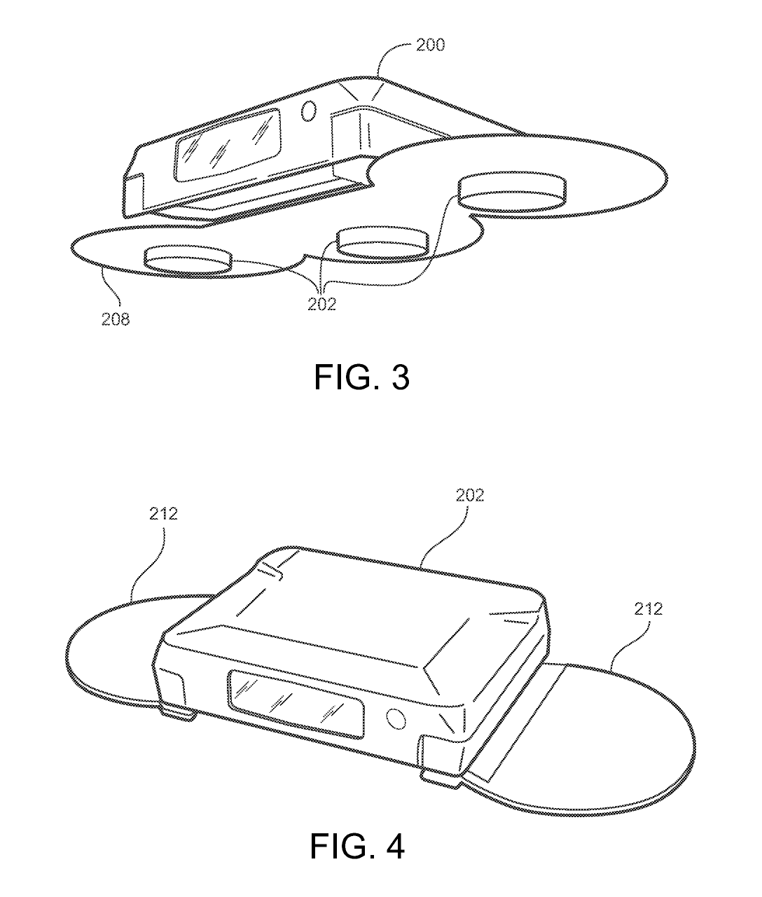

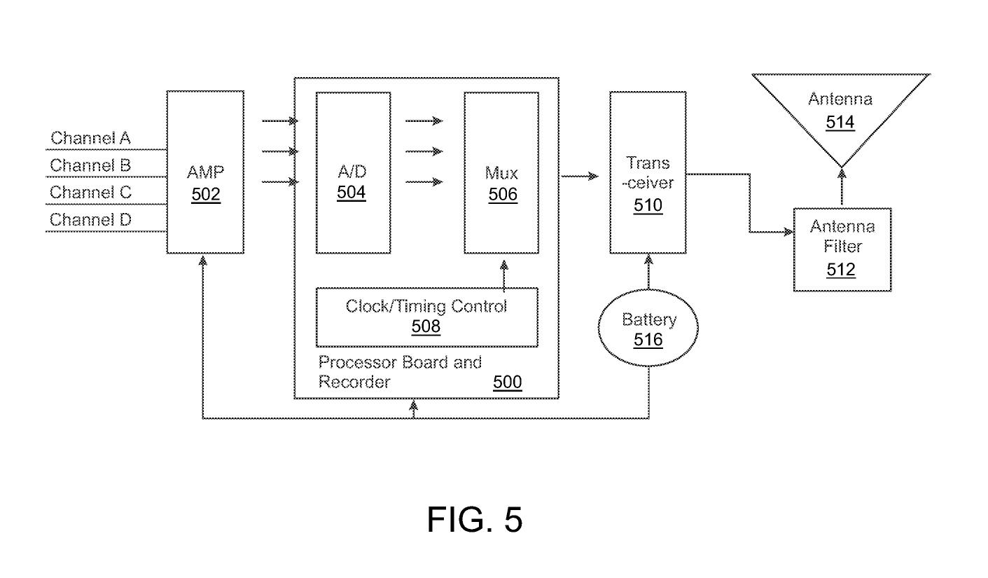

FIGS. 3 and 4 illustrate bottom and top perspective views, respectively, of the system shown in FIG. 2.

In accordance with embodiments, a monitoring system does not have the capability to generate ECG tracings, nor does the device perform any ECG analytical functions. The monitoring system may transmit the data via a communication system to the analysis station in formats that conform to the Institute of Electrical and Electronics Engineers (IEEE) 801.11a & b & g specifications. The communication system can be determined by the end user.

FIG. 7 illustrates a block diagram of another example medical data acquisition system for monitoring and diagnosis in accordance with embodiments of the present subject matter. Referring to FIG. 7, the system is a 4 channel system in which channels A, B, C, and D are electrically connected to 4 electrodes of a patch (not shown). Although 4 channels are shown in this example, it should be understood that the system may include any number of channels connected to a corresponding number of electrodes on one or more suitable electrode patches. The patch may be one of the patches as shown and described by the examples herein, or any other suitable electrode patch.

The monitoring system may include a suitable 2 to 8 channel ECG processing board and recorder 500. The monitoring recorder stores and transmits data received from the processing board via a communication system to a remotely located ECG analysis station for evaluation by a medical professional. Particularly, the board and recorder 500 may include an amplifier 502 having inputs that connect to the channels A, B, C, and D for suitable conditioning. The output of the amplifier 502 may be connected to an input of an analog-to-digital (A/D) converter 504, which is in turn connected to a multiplexer (MUX) 506. As an example, the A/D converter 504 may have a sampling rate greater than 1,200 s/s or any other suitable rate. The monitoring system is capable of performing various types of test such as, but not limited to, Holter monitoring, 24 hour continuous monitoring for event monitoring, vector cardiograms, arrhythmia monitoring, signal averaged ECG's, O₂ saturation, respirations and other patient physiological data depending on the type of analytical software used to interrogate and evaluate the processing board. The ways the signals are processed from the human body are both unique and advanced. The use of various amplifier and filtering techniques are used to increase the overall signal-to-noise ratio (SNR). The data set from the human body that the device is capable of processing may also be varied depending on the external software. Some of the examples of the type of data that can be acquired and processed by the card are: EKG, EEG, temperature, respirations, oxygen saturation, and the galvanic skin resistance. The processing board and monitoring recorder 500 can use various techniques to increase the SNR and thereby remove most muscle noise and outside environmental noise to produce a cleaner and over all better signal for the post processing software to use.

A clock/timing control module 508 may control timing of the MUX 506. A transceiver 510 may receive the output of the MUX 704 and wirelessly communicate physiological data or other data via an antenna filter 512 and an antenna 514. A battery 516 may power the system components.

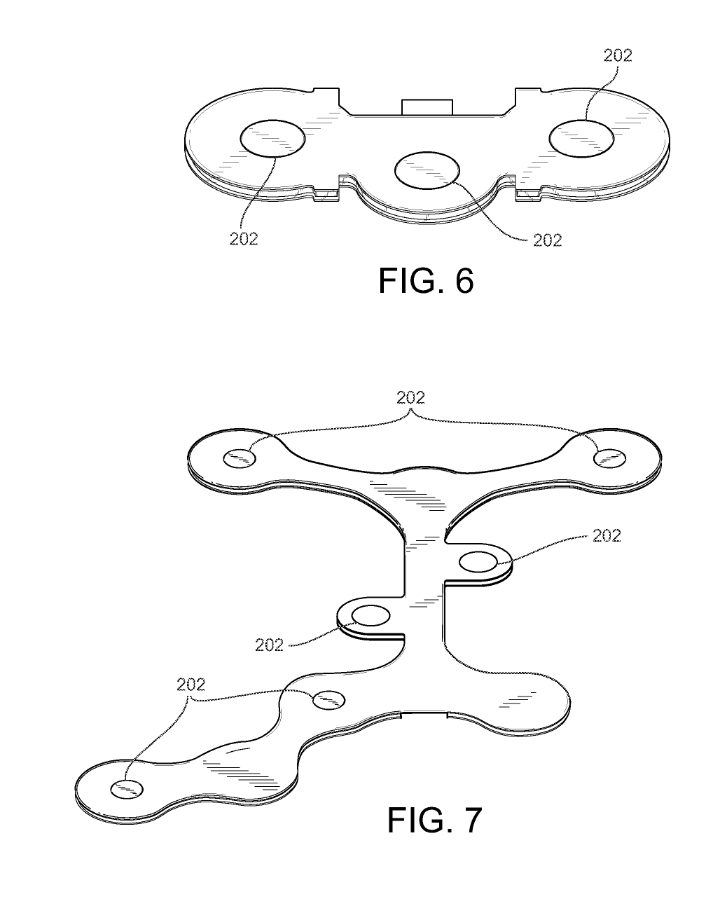

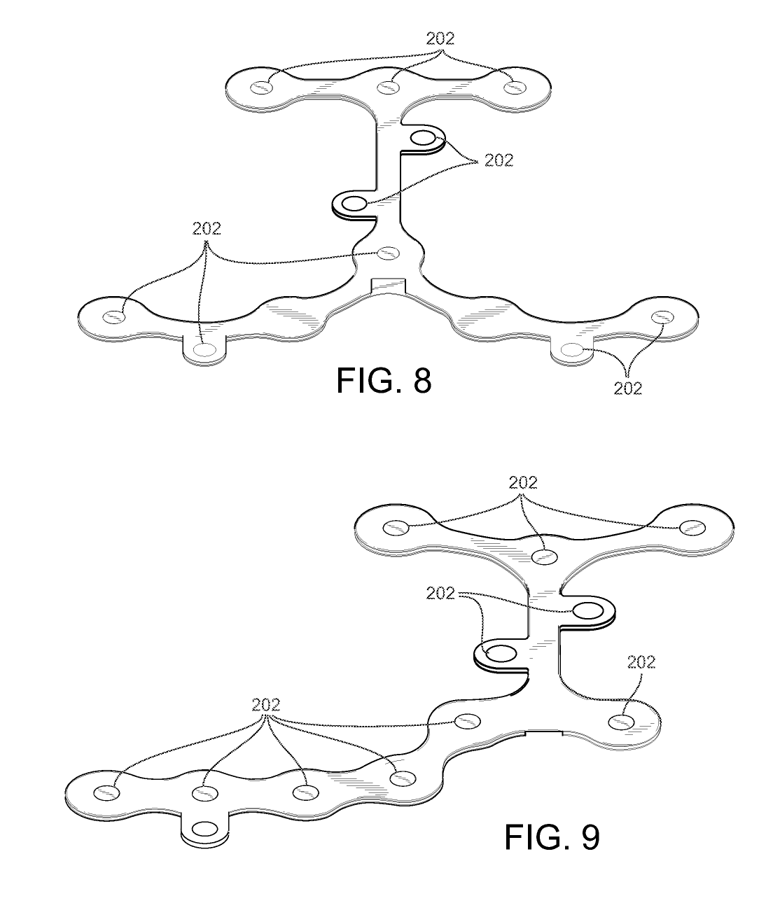

FIGS. 6-9 illustrate different examples of patch electrodes 600 in accordance with embodiments of the present subject matter. The patch electrodes 600 may be used with any of the systems and methods disclosed herein. Referring to FIGS. 6-9, the patch electrodes 600 may each be a multi-layer patch including embedded ECG electrodes 202, leads (not shown), and a battery (not shown) on a Mylar substrate. The patch electrode 600 can be affixed to a patient's chest for monitoring ECG activity or other physiological activity. The patch electrode 600 can have various designs depending on analysis software. For Phillips, it includes four precordial ECG electrodes that are positioned orthogonally so that, when the patch is applied in accordance with the present subject matter, the leads correspond to positions of EASI & G. The patch electrode may be used with modified Frank algorithms or any other suitable algorithms and techniques. Other suitable patch designs may be set up to various software configurations including the MEANS algorithm or other suitable algorithms and techniques. The patch component, though varying in configurations, can be divided into layers, which includes a top layer of polyester biocompatible foam with a lower adhesive layer. The middle layer can be a mylar-based conductive layer with adhesive covering both sides. The bottom layer is the same as the top layer with the use of a polyester-based reticulated foam to allow for a conductive gel to be utilized which can provide a medium between the human body and the middle layer conductive surface.

The ECG leads in all models of the patch electrode are connected to the processing board. The processing board receives the ECG data from the leads and transmits or stores this information to the monitor recorder.

The various techniques described herein may be implemented with hardware or software or, where appropriate, with a combination of both. Thus, the methods and apparatus of the disclosed embodiments, or certain aspects or portions thereof, may take the form of program code (i.e., instructions) embodied in tangible media, such as floppy diskettes, CD-ROMs, hard drives, or any other machine-readable storage medium, wherein, when the program code is loaded into and executed by a machine, such as a computer, the machine becomes an apparatus for practicing the presently disclosed subject matter. In the case of program code execution on programmable computers, the computer will generally include a processor, a storage medium readable by the processor (including volatile and non-volatile memory and/or storage elements), at least one input device and at least one output device. One or more programs may be implemented in a high level procedural or object oriented programming language to communicate with a computer system. However, the program(s) can be implemented in assembly or machine language, if desired. In any case, the language may be a compiled or interpreted language, and combined with hardware implementations.

The described methods and apparatus may also be embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, a video recorder or the like, the machine becomes an apparatus for practicing the presently disclosed subject matter. When implemented on a general-purpose processor, the program code combines with the processor to provide a unique apparatus that operates to perform the processing of the presently disclosed subject matter.

Features from one embodiment or aspect may be combined with features from any other embodiment or aspect in any appropriate combination. For example, any individual or collective features of method aspects or embodiments may be applied to apparatus, system, product, or component aspects of embodiments and vice versa.

While the embodiments have been described in connection with the various embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed:

1. A system comprising:
one or more electrodes configured to detect physiological data and to convert the detected physiological data into a signal;
a monitor recorder configured to receive the signal and store the detected physiological data; and
a transceiver configured to wirelessly communicate the physiological data.

2. The system of claim 1, wherein the physiological data includes one of respiration data, oxygen data, and saturation data.

3. The system of claim 1, wherein the monitor recorder is configured to receive a plurality of signals from the one or more electrodes via a plurality of channels.

4. The system of claim 1, wherein the monitor recorder comprises an amplifier configured to amplify the signal.

5. The system of claim 1, further comprising a battery configured to power the monitor recorder and the transceiver.

6. The system of claim 1, wherein the physiological data includes electrocardiography (ECG) data.

7. The system of claim 1, wherein the monitor recorder is configured to receive one of sound data and accelerometer data, and
wherein the transceiver is configured to wirelessly communicate the one of the sound data and accelerometer data.

8. A method comprising:
detecting physiological data and converting the detected physiological data into a signal;
receiving the signal and storing the detected physiological data; and
wirelessly communicating the physiological data.

9. The method of claim 8, wherein the physiological data includes one of respiration data, oxygen data, and saturation data.

10. The method of claim 8, wherein detecting and converting comprises using one or more electrodes to detect the physiological data and to convert the detected physiological data into the signal.

11. The method of claim 8, wherein receiving the signal comprises receiving a plurality of signals from the one or more electrodes via a plurality of channels.

12. The method of claim 8, further comprising amplifying the signal.

13. The method of claim 8, further comprising powering the monitor recorder and the transceiver.

14. The method of claim 8, wherein the physiological data includes electrocardiography (ECG) data.

15. The method of claim 8, further comprising:
receiving one of sound data and accelerometer data; and
wirelessly communicating the one of the sound data and accelerometer data.
